(19) United States
(12) Patent Application Publication
(10) Pub. No.: US 2021/0087601 A1
(43) Pub. Date: Mar. 25, 2021

(54) A SPECIFIC, RAPID TEST DIFFERENTIATING GRAM POSITIVE AND GRAM NEGATIVE BACTERIA

(71) Applicant: HELIER SCIENTIFIC LTD, Surrey (GB)

(72) Inventors: Robert Graham PRICE, Hertfordshire (GB); Zahra HASSAN, Surrey (GB); Ramadan ABUKNESHA, London (GB)

(73) Assignee: HELIER SCIENTIFIC LTD, Surrey (GB)

(21) Appl. No.: 16/603,390

(22) PCT Filed: Apr. 4, 2018
(86) PCT No.: PCT/GB2018/050914

§ 371 Date: Oct. 7, 2019

(30) Foreign Application Priority Data
Apr. 7, 2017 (GB) 1705666.4

(51) Int. Cl. C12Q 1/04; G01N 33/558; G01N 33/569; C07K 16/12; B01L 3/00

(52) U.S. Cl. CPC C12Q 1/04; G01N 33/558; G01N 33/56911; B82Y 35/00; C07K 16/1267; B01L 3/502761; C07K 16/1203

(57) ABSTRACT

The present invention discloses a specific, rapid test differentiating microorganisms by trait, such as distinguishing gram positive and gram negative bacteria, in bodily fluids at point of care. This is achieved through a method of producing a polyclonal antibody targeted towards the particular trait of the microorganism to be tested for, as well as the polyclonal antibody produced by such a method. Also disclosed is a lateral flow point of care test device comprising such a polyclonal antibody, as well as a method of use of such a device.

### TECHNICAL FIELD

The present invention discloses a method for the rapid detection and differentiation of microorganisms by trait (such as gram positive/gram negative) in bodily fluids at point of care. In addition, it has other applications in the monitoring of water purification, the food and agriculture industries and field medicine. The invention would allow the administration of the correct antibiotic at point of care, avoiding overuse and incorrect prescription.

### BACKGROUND ART

Characterisation of bacterial infection is currently performed using traditional laboratory culture methods which take a minimum of several days to complete. Clinicians may have to wait up to a week for definitive results so that they can determine the correct antibiotic to administer. The few rapid methods currently available are expensive, may require sophisticated equipment and technical expertise. This results in antibiotics often being administered before confirmatory results are available, which may therefore be inappropriate or even harmful to the patient.

The need for a rapid point of care test is emphasised when the emergence of drug resistant bacteria as a global phenomenan is considered. This poses a huge threat to human and animal health. The Chief Medical Officer (UK) has gone as far as stating that antibiotic resistance is a “catastrophic threat” equal to terrorism and climate change. Thousands of people currently die every year in Europe alone due to infections resistant to antibiotic drugs and the numbers are much greater worldwide. This global increase of bacterial resistance to antibiotics threatens to return medicine and surgery to the pre-1940 era where minor infections could be fatal. Inappropriate prescription of antibiotics together with their overuse is one of the major contributions to the rapid rise of antibiotic resistance. A recent report by The National Institute for Health and Care Excellence estimated that there are 10 million unnecessary prescriptions issued for antibiotics in England per year—that is 1 in 4 of every prescription. Although new guidelines may be implemented which could see General Practitioners facing disciplinary action for over-prescribing antibiotics they have no suitable point of care test available to them for correctly determining when antibiotics are needed and what type.

When testing an unknown sample of microorganisms, it is common practice in microbiology to determine whether the organisms are gram positive or gram negative as the very first step in the protocol of identification. Following this, further tests and investigations are carried out to determine which sub group the organism belongs to until the specific organism is identified. There has been much work and interest in the area of rapid determination of specific organisms using molecular methods and other techniques. However, to date the determination of the gram status of the microorganism as a point of care test has received little attention.

WO 2010/077268 A1 describes a quantitative strip analyte assay device and method for determination of a number of different analytes including bacteria, viruses and fungi. Semi-quantitation was achieved using two known calibrators.

US 2010/0129836 A1 discloses a system for detecting bacteria in blood, blood products, and fluids of tissues. This procedure is able to detect clinically relevant levels of bacteria blood, blood products and body fluids using a variety of immunoassay formats. The embodiment of the tests was varied to detect either gram positive or gram negative bacteria or both together.

WO 2012/078426 A2 describes a filter device which is able to concentrate bacteria shortening or avoiding culture time in clinical, food, environmental or other samples. The device could be modified to concentrate on specific organisms.

WO 2013/049596 A2 describes immunoassay procedures for the detection, identification and quantification of gram negative bacteria.

U.S. Pat. No. 9,434,977 B2 discloses a lateral flow assay that can provide an indication of Gram negative (GN) or Gram positive (GP) infection (or both) within 30 minutes. It reveals a number of problems with existing lateral flow assay devices for distinguishing said bacteria. Firstly, there is a risk of cross reaction as a result of the use of commercially-obtained antibodies with proteinase utilised in their production which may yield false positives. Secondly, the change in colour of the test line on the lateral flow strip can be extremely faint, with the result that a special device is preferably used in order to assess whether detection has occurred in the first place, making the system substantially more awkward and expensive to use.

There is therefore a long-felt need for a detection method which shows a heightened response to the traits in question.

### SUMMARY OF INVENTION

The present invention provides a novel method for the production of polyclonal antibodies targeted towards a particular trait expressed by microorganisms, such as gram positivity or gram negativity. The enhanced response of these novel and inventive antibodies for that trait in particular means that more clear and unambiguous results are obtained when said antibodies are used in detection processes. Various preferable variants of this method are disclosed in the claims.

The present invention also provides a device for the detection of traits of microorganisms that such polyclonal antibodies have been produced for, and which utilises said polyclonal antibodies. For instance, using the method of the present invention we can produce a device for the detection of microorganisms and the characterisation of the gram status of the microorganisms comprising gram specific antibodies able to distinguish gram negative and gram positive organisms. The applicants have discovered that there is an unmet need for such a test and no such test exists on the market to date. Various preferable variants of such devices are disclosed in the claims.

The invention is exemplified by a device based on a lateral flow format. A typical lateral flow consists of four components: sample application pad, conjugate release pad, the analytical nitrocellulose membrane and the absorption wick (FIG. 1). Each section will have different characteristics such as absorption, wicking rates and binding properties thereby allowing a good flow through and clear results. The lateral flow format is generally used in the detection of compounds by antibodies and its main advantages are ease of use and speed. The device is designed for the detection and characterisation of gram positive and negative bacteria.

Antibodies can be used to optimise the conditions and components in the lateral flow immunoassay format. The lateral flow system can utilise a range of different papers and buffer systems which need to optimised to enable the most sensitive system to be used. The specificity of all antibodies can be determined using enzyme linked immunosorbent assay (ELISA). Gold nanoparticles (sizes 20 nm and 40 nm) can be obtained e.g. from BBI Solutions Ltd and can be conjugated to the gram positive and gram negative antibodies. Prior to use in the lateral flow format they need to be tested on nitrocellulose paper using the dot blot procedure. Various bacteria dilutions can be used with a range of antibody concentrations to determine optimum conditions for successful gold conjugation.

This example device is a novel and innovative Point of Care test device which will detect and differentiate between groups of bacteria. Antibiotics are generally prescribed based on the group of bacteria causing infection as opposed to the specific organisms. The proposed test will enable doctors to make a decision within minutes whether an antibiotic is needed and therefore which type is appropriate. This will enable the correct prescription to be made, thereby reducing the number of antibiotics used as well as improving patient care and saving costs. The test can also be developed for use in other diagnostic platforms and incorporate antibiotic susceptibility tests. It can be readily adapted for use in veterinary medicine, developing countries, battlefields, agriculture in farmed animals, fish farming and the food industry.

### BRIEF DESCRIPTION OF THE DRAWINGS

The illustrations included are described below:

FIG. 1: A schematic of a lateral flow strip construct developed during the preliminary studies in accordance with the invention.

FIGS. 2a and 2b: Examples of the various potential formats of the final Lateral flow strip device in accordance with the invention.

### DETAILED DESCRIPTION

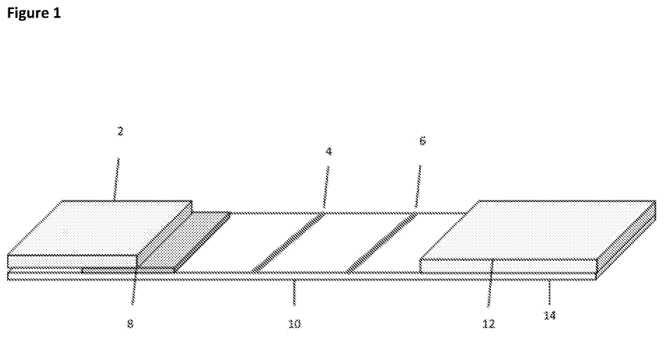

FIG. 1 shows a preferred embodiment of the lateral flow point of care test device of the invention. A sample of microorganisms is administered to a sample application pad 2 containing a buffer which is at least partially overlapping the conjugate release pad 8 infused with a suitable antibody. The sample is drawn from the sample application pad 2 through the conjugate release pad 8 and through a nitrocellulose membrane 10, which is at least partially attached under the conjugate release pad 8. At the other end of the nitrocellulose membrane 10 to the sample application pad 2 and conjugate release pad 8 is an absorbent pad 12 which is responsible for drawing the sample from one end of the device to the other.

As the microorganisms in the sample are drawn through the conjugate release pad 8, any gram positive or negative bacteria, depending on which the particular device is testing for, bind to the antibodies infused therein. Travelling further, the first antibodies bind to a second set of antibodies located in a test line 4 on the nitrocellulose membrane 10, which accordingly becomes visible, indicating that the sample contains gram positive or gram negative bacteria, depending on which antibodies the test line contains. The device further comprises a control line 6, which indicates whether or not the test has been successful, and a backing 14, upon which the other elements of the device are situated.

Prototyping work was performed with an aim to produce a preferable device for use in a strip test able to distinguish between gram positive and gram negative bacteria using a lateral flow gold coupled antibody procedure. The objective was to obtain visible coloured lines minutes after direct application of a pathological sample.

Escherichia coli and Pseudomonas aeruginosa were used as examples of gram negative organisms while Staphylococcus aureus, Bacillus subtilis and Streptococcus agalactiae were used as examples of gram positive organisms. These were sourced from The National Collection of Type Cultures (NCTC), UK and were cultured in LB broth and Nutrient agar (Sigma-Aldrich, UK) for a few generations following which stocks were prepared and stored at 4° C. and −70° C.

The antibodies selected were chosen for their suitability for use in strip assay procedures and their availability (Table 1). They were either monoclonal or polyclonal antibodies raised against unique components of the surface of either gram positive or gram negative bacteria. The characteristics of the chosen antibodies were initially assessed using ELISA (enzyme linked immunoassay) and Dot blot techniques.

TABLE 1

| Antibody | | | Product |
|---|---|---|---|
| Description | Abbreviation | Company | code |
| Anti-gram positive bacteria | Mm AntiG+ | Abcam | Ab20344 |
| antibody [BDI380]. Mouse | (ab20344) | | |
| monoclonal Anti-LTA. 1.8 mg | | | |
| Anti-gram positive bacteria | MmAntiG+ | Abcam | Ab155857 |
| antibody [3801]. Mouse | (ab155857) | | |
| monoclonal. 0.1 mg. Anti-LTA | | | |
| Mouse anti gram positive | MmAntiG+ | Acris | BDI813 |
| bacteria BDI813 - 1 mg. Anti- | (Acris-BDI813) | Antibodies | |
| LTA | | | |
| Mouse anti-gram negative | Mm AntiG− | Abcam | Ab31204 |
| bacteria monoclonal antibody, | (ab31204) | | |
| reacts with endotoxin-2 mg/ml. | | | |
| Anti-gram negative endotoxin | MmAntiG− | Abcam | Ab20954 |
| antibody [306] -mouse | (ab20954) | | |
| monoclonal-ascites fluid | | | |
| Lipid A LPS Endotoxin | GpAntiG− | Thermo | PA1- |
| Antibody (Pierce) - Goat | (Pierce) | Fisher | 73178 |
| Polyclonal -4-5 mg/ml | | | |
| Lipopolysaccharide Gram | GpAntiG− | Acris | BP2235 |
| negative antibody - Polyclonal- | | Antibodies | |
| Goat-BP2235-4-5 mg/ml | | | |

The optimum conditions for the interaction of the above antibodies with the selected bacteria were established using ELISA. In order to achieve this, a wide range of concentrations of bacteria were used which replicated the concentrations of bacteria found in pathological samples. Once the optimum concentrations were identified, the antibodies were then tested using Dot Blot technology in order to optimize the antibody reaction conditions on nitrocellulose paper.

A range of commercially available papers were tested in the prototype strip to achieve the optimum results. The lateral flow strip consisted of 4 components—a nitrocellulose membrane, a sample application pad, a conjugate pad and an absorption pad (FIG. 1).

TABLE 2

| Sample Paper | Potential Strip Component |
|---|---|
| Whatman Rapid 27 | Sample application |
| Whatman Rapid 24 | Pad |
| Whatman STD 17 | Conjugate Pad |
| Whatman STD 14 | |
| Whatman Rapid 27 | |
| Whatman FUSION 5 ™ | |
| Immunopore RP | Nitrocellulose Membrane |
| Whatman CF6 | Absorbent Pad |

In addition, the following paper present from previous studies was also investigated.

TABLE 3

| Sample Paper | Potential Strip Component |
|---|---|
| Pall Cellulose Absorbent Pad 165 | Absorbent Pad |

Below are the properties of the various papers investigated for the different components of the prototype strip:

Sample Application Pad:

Rapid 24 and 27 were initially investigated for use as a sample application pad. They are made up of treated bound glass fiber material which has good rewetting properties and improved conjugate release without interfering with assay sensitivity.

TABLE 4

| | Thickness | Wicking Rate | Water Absorption |
|---|---|---|---|
| Paper | (μm @ 53 kPA) | (s/4 cm) | (mg/cm²) |
| Rapid 24 | 340 | 38 | 55 |
| Rapid 27 | 365 | 39 | 40 |

Conjugate Pad:

Standard 14 and 17 are untreated grades of bound glass fiber material and are suitable for conjugate pad optimization, particularly for sensitive assays. They have a faster flow than cotton and lower sample retention, thus making them a likely choice for the conjugate release pad.

Rapid 27 has been described above and was selected to be investigated as a conjugate pad due to its ability to release more conjugate than untreated pads.

The FUSION 5™ is a unique type of paper developed by Whatman which is a single material that performs all the functions of a lateral flow strip. It's designed to be used in a wide range of tests simplifying manufacturing and reducing costs.

TABLE 5

| | Thickness | Absorption | Max. | % Release of |
|---|---|---|---|---|
| | (μm @ | Capacity | Pore size | Gold Conjugate |
| Paper | 53 kPA) | (mg/cm²) | (μm) | (after 90 secs) |
| Standard 14 | 355 | 55 | 23 | 75 |
| Standard 17 | 370 | 35 | 23 | 75 |
| Rapid 27 | 365 | 40 | 22 | 86 |
| FUSION | 370 | 40 | 11 | >94 |
| 5 ™ | | | | |

Nitrocellulose Membrane:

The Immunopore membrane is the preferable choice for the most sensitive assays, particularly assays for infectious disease, therefore, was the obvious choice for use with this strip prototype. It offers the best reproducibility, stability and accuracy and is available in three wicking rates. Only the Immunopore RP, the general purpose membrane, was investigated in this study.

TABLE 6

| | Capillary Rise | Caliper |
|---|---|---|
| Paper | (s/4 cm) | (μm) |
| Immunopore RP | 85-115 | 200 |

Absorbent Pad:

CF6 is made up of a mixture of cotton and glass fiber and was selected as a possible material for the absorbent pad because of its unique property to minimize any conjugate backflow. In many assays, the sample and conjugate tend to run back up the membrane after the reaching the absorbent pad which can result in false positives.

Pall 165 membrane was also assessed because of its thickness and high absorption properties. It has been used as the absorbent pad in other tests developed by the inventors and it also successfully reduces backflow.

The Pall 165 proved to be a better absorbent and is therefore preferable for use as the absorbent pad in the final strip prototype. The CF6 was preferred as the sample application pad as its thickness and water absorption was greater than Rapid 24 and 27, allowing the sample volume to be applied with more ease. Out of the papers investigated, the FUSION 5™ proved to be the most superior paper for the conjugate pad, due to its high percentage release of gold conjugate, allowing the maximum amount of gold coupled antibody-bacteria complex to move to the test and control lines. The nitrocellulose Immunopore RP membrane was satisfactory for its purpose, however, there is potential to investigate the other two wicking rates.

The papers finally selected for the various segments of the preferred prototype construct are listed in the Table 7 below:

TABLE 7

| Strip Prototype | |
|---|---|
| Component | Specification |
| Sample application pad | CFS glass fibre/cellulose paper obtained |
| (1.5 × 1 cm) | from Whatman International Ltd., Uk. The |
| | paper was pretreated with Borate buffer, |
| | pH 7.5 containing 1% (w/v) BSA, 0.5% (v/v) |
| | Tween 20 (RTM) and 0.05% (w/v) sodium |
| | azide, dried at 37° C. for 2 hours before |
| | assembly. |
| Conjugate pad | FUSION 5 ™ pad (Whatman International |
| (1 × 1 cm) | Ltd., UK) immersed in conjugate suspension |
| | and dried at 37° C. for 2 hours before |
| | assembly. |
| Nitrocellulose membrane | Immunopore RP paper (Whatman |
| (2.5 × 1 cm) | International Ltd., UK), untreated. |
| Absorbent pad | PALL 165 Paper (obtained from Pall Europe |
| (1.7 × 1 cm) | Ltd., Portsmouth, UK), untreated. |

Optimising the Size of Gold Particles

The binding of the primary commercial antibody (e.g., mouse anti-gram positive monoclonal antibody against lipo-tecichoic acid, MmAb αGr+ve, 50 μl/ml, Abcam plc) with 40 nm colloidal gold particles (BBI solutions) in 2 mM borate buffer pH 9.0 was unsuccessful as none of the conjugates contained enough antibody adsorbed onto the gold nanoparticles to prevent floculation when 10% sodium chloride was added. Similar experiments with 20 nm gold nanoparticles (BBI Solutions, Cardiff) when Mouse anti-gram positive monoclonal antibody (MmAb αGr+ve, Abcam) concentrations up to 60 μg/ml were added again resulted in flocculation. Further experiments were carried out in this case using gold nanoparticles (40 nm) with goat anti-lipid A LPS endotoxin Gram −ve polyclonal antibody (GpAb αLipid A, 4-5 mg/ml, Pierce Thermo) following removal of sodium azide (Abcam.com/technical). Aliquots (100 μl) of different dilutions (10-200 μl/ml) of the GpAbaLipidA antibody were incubated with 1 ml of 40 nm 0.15 nM gold nanoparticles (AuNp₄₀ₙₘ) and added to 100 μl aliquots and left for 2 min with gentle mixing, no flocculation chloride was found with 10% sodium when antibody concentrations 20, 50 and 200 μg/ml were used. Gold particles of either 20 nm or 40 nm in diameter were used in the final construct.

All the antibodies used were conjugated with gold nanoparticles either directly using 2 mM borate buffer pH 9.0 or using a commercial kit (Innova Biosciences Ltd). Antibodies were applied to the conjugation pad of the strip. The test and control lines were situated on the nitrocellulose membrane. The bacterial or pathological fluid sample(s) were loaded on the sample application pad. Movement along the strip was due to capillary action and driven by the absorption pad. The detection of the bacteria was achieved using either monoclonal or polyclonal antibodies located at the test lines. The validity of each test was established using polyclonal antibodies located on the control line. The sensitivity of the strip was assessed using clinical ranges of gram positive and gram negative bacteria.

Table 8 below summarizes the performance of the seven commercial antibodies investigated.

TABLE 8

| | | | Gold | |
|---|---|---|---|---|
| Antibody | ELISA | Dot Blot | Conjugation | Lateral Flow |
| Mm AntiG+ | − | − | − | − |
| (ab20344) | | | | |
| MmAntiG+ | + | + | + | +/− |
| (abl55857) | | | | |
| MmAntiG+ | + | +/− | + | − |
| (Acris-BDI813) | | | | |
| Mm AntiG− | − | − | − | − |
| (ab31204) | | | | |
| MmAntiG− | + | +/− | + | +/− |
| (ab20954) | | | | |
| GpAntiG− | + | + | + | + |
| (Pierce) | | | | |
| GpAntiG− | + | + | + | − |
| (Acris) | | | | |

The major challenge posed was finding a suitable, good quality commercial antibody. Some of the antibodies were reacting with both gram positive and gram negative organisms and as a result lowered the sensitivity of the test.

Polyclonal Antibodies Selecting for a Specific Trait

The method of obtaining polyclonal antibodies of the present invention overcomes the shortcomings of commercially obtained prior art antibodies, thus enabling the production of a more sensitive test.

Essential to the procedure is the availability of robust antibodies which are meticulously selected to recognise their target antigens in the lateral flow format. Although the physical components of the test strip, construction techniques and buffers play the major role in optimizing the test, the heart of these processes are the functional properties of antibodies, which need to be carefully designed at the stage of selecting antigens for immunisation and highly purified. Antibodies which perform well in e.g. ELISA may not always be suitable in lateral flow test format immunoassay. The applicants propose a novel approach to synthesising the generation of anti gram negative and anti-gram positive antisera antibodies. Teichoic acid is specific to gram positive organisms and is available from Sigma Aldrich or an equivalent supplier. Teichoic acid from 4 or more different types of gram positive bacteria can be mixed and used as gram positive biomarker antigens. The mixed antigen can then be coupled to a suitable carrier immunogenogenic protein such as keyhole limpet hemocyanin (KLH) to form an immunogen. Similarly, Lipopolysaccharide (LPS) is specific to gram negative organisms and can be obtained from Sigma Aldrich or equivalent supplier. LPs preparations derived from four or more different gram negative bacteria can be mixed and can be used as gram negative biomarker antigens and following coupling to a suitable carrier protein form the immunogen.

Commercial Teichoic acid and LPS can be contaminated with traces of the other bacterial biomarker (e.g. traces of LPS in teichoic acid preparations and vice versa), which is the principal cause of cross reactivity of the resultant antisera. In order to enhance the specificity of the generated antisera, it is intended to purify the commercial teichoic acid and LPS by e.g. SDS-PAGE electrophoresis prior to subsequent steps. The chemical coupling (conjugation) of the gram positive and gram negative biomarker antigens to carrier proteins will be carried out using methods that avoid masking the critical unique moieties of the bacterial antigens and obviate extreme alteration of the bacterial biomarker molecules. The immunogens can be injected into sheep or other appropriate species in order to generate polyclonal antibodies. The serum from sheep or other appropriate species should be assessed every month, up to 6 months. The affinity of the antibodies towards the antigenic sites on both gram positive and gram negative bacteria can be assessed in the serum obtained at 3 months and 6 months post injection. When high affinity has been demonstrated in the serum the antibody can be further purified using salt precipitation and affinity purification enabling the antibody concentration, activity and titre to be determined.

Polyclonal antibodies produced according to the preceding method can be incorporated into a lateral flow immunoassay paper strip construct. The efficacy of the antibodies can be assessed using bacteria grown in broth, saline and urine or other body fluid samples spiked with bacteria. The construct and all its constituents are maximised to achieve best results.

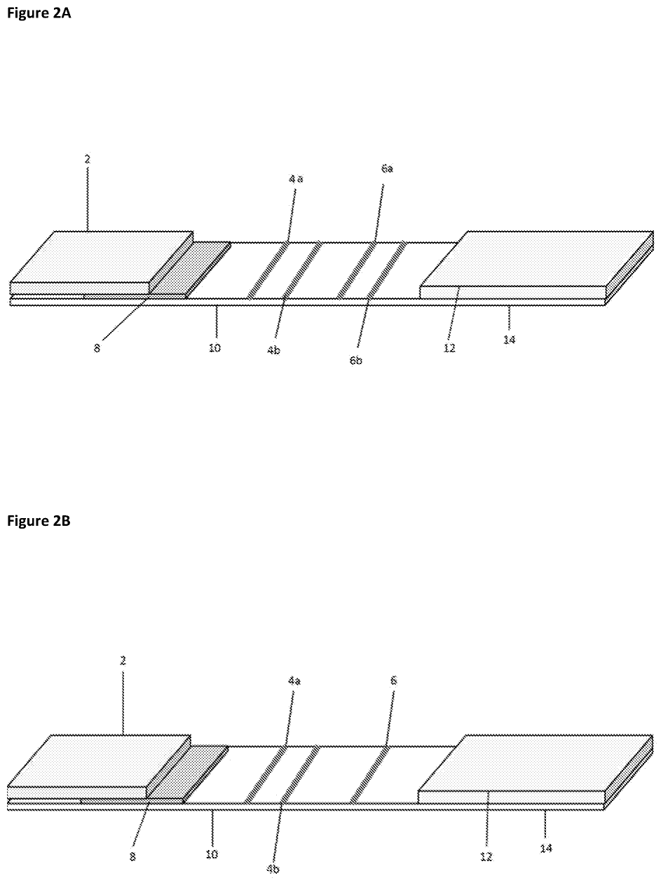

A number of different strip constructs are possible, as shown in FIGS. 1, 2A and 2B. For example, FIG. 1 shows one type which is designed simply for either gram positive bacteria or for gram negative bacteria, but only one of these at a time. FIG. 2 shows another type which can detect the presence of both gram-positive and gram negative bacteria simultaneously. Instead of having one test line 4 as the device in FIG. 1 does, this embodiment has two test lines 4a and 4b. As the sample travels through the conjugate release pad, gram positive bacteria bind to the antibodies which target teichoic acid, whilst gram negative bacteria bind to the antibodies which target lipopolysaccharides. One of the test lines 4a and 4b will contain secondary antibodies which target one of the primary antibodies, whilst the other test line will contain secondary antibodies which target the other primary antibody. The test lines 4a and 4b will therefore become respectively visible when gram positive or gram negative bacteria travel through them.

The system of FIG. 2 can have alternative designs depending on the control line(s) 6, as shown in FIGS. 2A and 2B. FIG. 2A has two control lines 6a and 6b, one corresponding to each of the two test lines 4a and 4b, whilst FIG. 3B has only one control line 6 acting as a control for both test lines 4a and 4b.

The lateral flow immunoassay device can be used at the point of care (POC) to establish bacterial groups present in the urine of selected cohorts of patients. The specificity and sensitivity of the test can be compared with the results obtained in the laboratory using standardised culture methods. Different formats of the strip tests, and different selections of strip components, can be used depending on the type of patients and body fluid used. The results obtained at point of care will allow the clinician to recommend the correct antibiotic to be used. The final aim will be to have a working single strip prototype, which is able to distinguish between infections caused by gram positive or gram negative bacteria of sufficient clarity to allow the administration of the correct antibiotics at point of care.

Other Potential Uses of the Test

The initial characterisation of infection into either gram positive or gram negative bacteria is crucial in enabling clinicians to administer the correct type of antibiotic and to start treatment. It can be envisioned that other traits could be tested for by the test instead of gram positive or gram negative status, simply by the same method of collecting antigens distinctive to said trait, producing therefrom a mixed antigen, and utilising the mixed antigen to produce a polyclonal antibody and creating a strip test from said antibody. Such traits could include cell morphology, class, order, family, genus, or species. Testing for such traits may be particularly useful in characterising miscellaneous organisms which do not stain well with gram stain tests; examples of these include Mycoplasma, Mycobacteria and Helicobacter.

The technology described could be modified to allow secondary more specific identification of the bacteria present in either group, by the introduction of species-specific antibodies into secondary strip tests. These would be a valuable addition to the point of care tests available in, for example, hospital infections. For example, the strip construct could be modified using antibodies to detect the common Gram positive bacteria; Staphylococci aureus (MRSA), Clostridium difficile, Streptococci or Enterococci. Alternatively, when Gram negative infections have been identified modified strip tests for the detection of E. coli, Pseudomonas aeruginosa, Klebsiella pneumoniae, Enterobacter or Proteus could provide more specific information.

When such secondary strip tests are available, this can then enable a “tiered” testing process, in which a first test takes place using a strip according to the present invention to determine the presence of gram-negative or gram-positive bacteria (or, conceivably, neither) in a sample. After this initial test, secondary tests for specific species (for example, with secondary strip tests) can take place, taking into account the result of the initial test.

For instance, table 9 outlines types of fluid that may be extracted in a testing procedure from the human body or from other sample sources, and families of gram positive or gram negative bacteria which may be especially likely to be found there; being able to positively identify (or rule out) the species of bacteria at this stage could be extremely helpful in ensuring that inappropriate antibiotics are not overprescribed and effective, species-specific treatment is provided.

Conversely, if the initial test suggested neither the presence of gram-positive or gram-negative bacteria, this could prompt explorations of other possibilities such as viral infection, sparing the use of antibiotics altogether. Such tiered tests would also be particularly advantageous in e.g. developing countries and areas of conflict where no laboratory facilities are available.

TABLE 9
Common infections in accessible body fluids or other sample sources

| | Type of | Gram | Gram |
|---|---|---|---|
| | fluid | positive | negative |
| 1 | Peritoneal dialysis | Staphylococcus | |
| | fluid | epidermis | |
| 2 | Urine | Enterococci (14%) | E. coli (64%), |
| | | | Coliforms (12%) |
| | | | including Klebsiella |
| | | | pneumoniae, Proteus (5%) |
| 3 | Wound fluid, | Staphylococcus | Pseudomonas aeruginosa |
| | exudates (ulcers) | aureus | (chronic wound infections) |
| 4 | Blood via venous or arterial | | Acinetobacter baumannii (80%) |
| | catheters in Intensive Care | | |
| | patients | | |
| 5 | Swabs - | Streptoccal | |
| | Throat infections | pharyngitis | |
| 6 | Blood, Bronchoscopy | | Klebsiella pneumoniae |
| | (lung) | | |
| 7 | Methicillin- | Staphylococcus | |
| | resistant Staphylococcus | aureus | |
| | aureus (MRSA) | | |
| 8 | Cystic fibrosis | | Bukholderia cepacia |
| 10 | Catheter fluid infections | | E. coli and Klebsiella |
| | e.g. bladder and | | |
| | intravenous catheters | | |
| 11 | Lacrimal fluid (tears) | Staphylococcus | |
| | | aureus, | |
| | | streptococcal | |
| | | pneumoniae | |
| 12 | Milk -Mastitis (cows) | | Coliforms |
| | a (severe) | | (E. coli and Klebsiella) |
| | Milk -Mastitis (cows) | Staphylococcal | |
| | b (less severe) | | |
| 15 | Food (animal general) | | E. coli |
| | Poultry | | Salmonella strains |
| 16 | Vegetables | Clostridium perfringens | |
| | Contamination (hands) | Staph. aureus | |
| 17. | Environmental water | | E. coli, Enterococci |

1. A method of producing a polyclonal antibody targeted towards a particular trait of a microorganism, comprising the steps of:
a) obtaining antigens specific to said trait from multiple different types of microorganisms expressing said trait;
b) mixing said antigens to produce a mixed antigen;
c) coupling said mixed antigen to a carrier protein to produce an immunogen;
d) injecting said immunogen into an animal;
e) obtaining polyclonal antibodies targeted towards said trait from said animal.

2. The method of claim 1, wherein the trait is gram positivity and the antigen is teichoic acid.

3. The method of claim 1, wherein the trait is gram negativity and the antigen is lipopolysaccharide.

4. The method of claim 1, wherein the trait is cell morphology, class, order, family, genus, or species.

5. The method of claim 1, wherein the carrier protein is keyhole limpet hemocyanin.

6. The method of claim 1, wherein the animal is a mammal.

7. The method of claim 1, wherein the animal is a sheep, pig, or ape.

8. The method of claim 1, wherein the polyclonal antibody is purified through a process of salt precipitation and affinity purification.

9. A polyclonal antibody produced by the method of claim 1.

10. A lateral flow point of care test device comprising the polyclonal antibody according to claim 9.

11. The device of claim 10, further comprising monoclonal antibodies.

12. The device of claim 10, wherein the polyclonal antibodies are produced by the method of claim 2 and the device permits the characterisation of gram positive microorganisms.

13. The device of claim 10, wherein the polyclonal antibodies are produced by the method of claim 3 and the device permits the characterisation of gram negative microorganisms.

14. The device of claim 10, wherein some of the polyclonal antibodies are produced by the method of claim 2 and some are produced by the method of claim 3 and the device permits the characterisation and differentiation of gram positive and gram negative microorganisms.

15. The device of claim 11, further comprising:
a sample application pad containing a buffer;
a conjugate release pad containing the polyclonal antibodies;
a nitrocellulose membrane; and
an absorbent pad.

16. The device of claim 15, wherein the application pad is CF6 glass fibre/cellulose paper pre-treated with borate buffer.

17. The device of claim 16, wherein the CF6 glass fibre/cellulose paper is pre-treated with borate buffer of pH 7.5, the borate buffer comprising:
1% (w/v) BSA;
0.5% (v/v) Tween 20; and
0.05% (w/v) sodium azide;
and being dried at 37° C. for 2 hours prior to assembly.

18. The device of 15, wherein the conjugate release pad has a maximum pore size of 11 μm.

19. The device of claim 15, wherein the conjugate release pad has a water absorption of 40 μm and a particle retention of 2.3 μm.

20. The device of claim 15, wherein the conjugate release pad is a FUSION 5 pad immersed in conjugate suspension and dried at 37° C. for 2 hours before assembly.

21. The device of claim 15, wherein the polyclonal antibodies in the conjugate release pad of the lateral flow device are conjugated to colloidal gold nanoparticles of 20 or 40 nm size.

22. The device of claim 15, wherein the nitrocellulose membrane is untreated Immunopore RP paper.

23. The device of claim 15, wherein the absorbent pad is untreated PALL 165 Paper.

24. The device of claim 15, wherein one end of the nitrocellulose membrane is attached under the absorbent pad and the other end is attached under the conjugate release pad, which is at least partially attached under the sample application pad.

25. The device of claim 15, further comprising a test line comprising a second set of antibodies.

26. A method of use of the device of claim 25, comprising the steps of:
administering a sample of microorganisms to the sample application pad;
the sample being drawn from the sample application pad through the conjugate release pad and through the nitrocellulose membrane towards the absorbent pad and past the test line;
such that the microorganisms in the sample bind to the polyclonal antibodies in the conjugate release pad as they are drawn through if they express the trait the antibodies target, and wherein if this occurs the antibodies from the release pad, now conjugated to the microorganisms in the sample, bind to a second set of antibodies in a test line on the nitrocellulose membrane;
the test line becoming visible when bound to by the conjugated antibodies and microorganisms, indicating that the microorganisms express the trait the antibodies target.
